(19) United States
(12) Patent Application Publication
(10) Pub. No.: US 2025/0268473 A1
(43) Pub. Date: Aug. 28, 2025

(54) DATA ACQUISITION SYSTEM FOR ACQUIRING PATIENT DATA AND PROCESS FOR ACQUIRING PATIENT DATA

(71) Applicant: Drägerwerk AG & Co. KGaA, Lübeck (DE)

(72) Inventor: Thomas GRAßL, Lübeck (DE)

(21) Appl. No.: 19/065,381

(22) Filed: Feb. 27, 2025

(30) Foreign Application Priority Data
Feb. 28, 2024 (DE) 10 2024 105 665.7

(51) Int. Cl. A61B 5/00; G16H 10/60; G16H 40/67

(52) U.S. Cl. CPC A61B 5/0024; G16H 10/60; G16H 40/67

(57) ABSTRACT

A data acquisition system (100) acquires patient data (105) of a patient (102) and includes a plurality of physiological sensors (110) for determining the patient data via electrodes (111a, 112a, 113a, 214a) and a data acquisition unit (130). At least two of the physiological sensors (111, 112) and/or adapter cables (371, 473) for the physiological sensor are signal connected to each other directly via a plug connection (115) and form a sensor group (116). The data acquisition unit includes at least one slot (132) for a plug connection (133) as a patient data interface. The patient data determined by the sensor group is output to the data acquisition unit via a common connector (120). The common connector is configured for a direct plug connection to the data acquisition unit, to a hub (250) for the data acquisition unit and/or to an adapter cable (371) for the data acquisition unit.

### CROSS REFERENCE TO RELATED APPLICATIONS

This application claims the benefit of priority under 35 U.S.C. § 119 of German Application 10 2024 105 665.7, filed Feb. 28, 2024, the entire contents of which are incorporated herein by reference.

### TECHNICAL FIELD

The invention relates to a data acquisition system for acquiring patient data. The invention also relates to a process for acquiring patient data of a patient.

### BACKGROUND

In principle, data acquisition systems are known for acquiring patient data. Typically, data from various sensors is fed to a patient monitor where it is collected and stored in a digital database. It is also known to merge patient data into combined data and evaluate it accordingly.

Against this background, DE 10 2007 045 140 A1 describes a mobile physiological data acquisition system for obtaining electrophysiological signals from a patient. Patient data is sent to a patient monitor via a data bus in order to enable joint evaluation and storage of this patient data

The use of wireless sensors for patient monitoring is also known. However, cable-based data transmission is preferred in everyday clinical practice due to its reliability and it enables continuous transmission in real-time.

### SUMMARY

It is an object of the present invention to provide an improved data acquisition system, in particular a data acquisition system that can be better adapted to a current patient situation.

According to a first aspect of the invention, a data acquisition system for acquiring patient data of a patient comprising a plurality of physiological sensors and a data acquisition unit is proposed for solving attaining this object.

The plurality of physiological sensors is configured to determine patient data by means of electrodes, wherein at least two physiological sensors of the plurality of physiological sensors and/or adapter cables for a respectively assigned physiological sensor of the plurality of physiological sensors are arranged and configured to be signal connected to each other directly (in terms of signal technology) via a plug connection and thereby form a sensor group. A sensor group configured according to the invention thus has at least two physiological sensors that acquire (capture) or generate patient data. The sensor group and/or the plug connection is set up to provide at least one measurement signal, preferably a plurality of measurement signals, and a reference signal of the electrodes. The or each measurement signal can be an analog or digital signal. The reference signal can be an analog or digital signal.

The physiological sensors can be provided as part of an ECG (EKG) system, EIT system or SEMG system.

The data acquisition unit is configured to have at least one slot for a plug connection as a patient data interface, wherein the patient data determined by the sensor group is output to the data acquisition unit via a common connector (common plug), and wherein the common connector is configured for a direct plug connection with the data acquisition unit, for a plug connection with a hub for the data acquisition unit and/or for a plug connection with an adapter cable for the data acquisition unit.

In the context of the invention, it was recognized that in everyday clinical practice, an increasing number of sensor cables in the vicinity of patients hinder work processes in everyday clinical practice and, for example, make it difficult to move or transport a patient. According to the invention, this problem is solved by optimizing a tree structure of cables in the immediate vicinity of the patient while retaining the measurement signal and reference signal. Thus, the provision of a sensor group with a common connector allows a reduction of slots and a reduction of cables.

For the purposes of this application, a tree structure is a structure in which several branches connect from one or more basic elements to connecting elements connected to the basic elements, resulting in the idealized shape of a tree or bush.

This has the advantage of reducing the effort involved in transporting a patient, in particular the number of plugs to be regrouped can be reduced. This can also reduce the risk of user errors in everyday clinical practice.

Furthermore, an optimized cable tree structure can reduce the amount of material required for patient treatment.

Finally, forming at least one sensor group according to the invention, which has at least two physiological sensors for determining patient data, allows the tree structure of cables to be changed quickly and easily. Such modularity of the data acquisition system enables a particularly advantageous rapid adaptation of the data acquisition system to a new treatment situation of the patient. In particular, the data acquisition system can be adapted quickly if the patient is transferred to or leaves the operating theater or intensive care unit, for example.

In the advantageous case of using patient cables, the modularity of the data acquisition system allows, for example, the number of leads used for the patient data to be changed at short notice.

In the context of the present invention, a physiological sensor (hereinafter also referred to in part simply as a sensor) is understood to mean a device with which a physiological measured variable of a patient can be detected, which is based on an electrical activity of the patient, for example of the patient's heart muscles, and provides this to obtain the patient data. A physiological sensor of this type is thus used to determine patient data comprising one or more measurement signals and a reference signal. Each individual sensor can perform an analog/digital conversion and/or output analog data. In this sense, a physiological sensor can also detect several different and/or similar measured variables of a patient. For example, a patient cable with a plurality of electrodes can be a single physiological sensor in the context of the present invention. In particular, it is possible that a reference potential, preferably analog, is transmitted as a reference signal between the individual sensors to provide the reference signal by means of the plug connection.

In the context of the present invention, an adapter cable is understood to be a cable that connects a first electronic component to a second electronic component. In this sense, an adapter cable can be an extension cable, a sensor cable and/or a cable that connects different types of connections to one another.

Patient data is data that is related to the patient. As such, patient data can include measured values such as body temperature, blood pressure, heart rate or the like, but also measured values to be further evaluated such as a measurement signal or reference potential as part of an examination using ECG, EIT, SEMG or the like. It is also conceivable that such patient data could include information such as the patient's name, a patient ID, a hospital department name and/or a bed identifier, for example a bed number.

For the purposes of the invention, a slot (a port-plug or slot/socket) can be a male or female part of a plug connection.

Preferred embodiments of the data acquisition system according to the invention are described below.

In a particularly preferred embodiment, the data acquisition unit is further configured to output the acquired patient data to a database via a wireless data connection. In this embodiment, the data acquisition unit advantageously forms a central interface for communication with the database. This allows the number of interfaces with the database to be reduced, thereby reducing the complexity of data acquisition in everyday clinical practice. The database is preferably stored on an external or internal memory. Wireless communication with the database can take place, for example, via a Bluetooth connection, a WLAN connection and/or the like. Alternatively or additionally, the data acquisition system according to the invention can provide a cable-based connection to the database. Preferably, communication with the database takes place via a hospital network.

In a further advantageous embodiment, the data acquisition unit is further configured to receive patient data from at least one physiological sensor from the plurality of physiological sensors via a wireless communication link. Such a wireless communication link can further reduce the number of cables in the patient environment

Preferably, the at least two physiological sensors of the wireless communication link comprise a communication interface to the database, the data of which can also be received with a certain time delay without jeopardizing the patient's health, such as during a diagnosis

Preferably, the at least two physiological sensors are set up to exchange and/or forward and/or provide a reference signal via the plug connection.

In a particularly preferred embodiment, the data acquisition system also has a hub which can be connected to the data acquisition unit via the at least one slot and which is configured to provide a respective plug connection to the sensor group, to a physiological sensor and/or to the adapter cable for a respectively assigned physiological sensor via a plurality of slots. The hub in this embodiment allows the tree structure of cables in the immediate vicinity of the patient to be further optimized. In this way, a number of cables can be reduced and/or a number of plug connections on the data acquisition unit can be reduced. In an advantageous variant of this embodiment, the hub also serves as an analog/digital converter for the patient data output by the physiological sensors. The hub may include one or more processors and a memory unit and may be configured for handling data packets and/or analog signals. A large number of different hub configurations are known to the skilled person, so that these will not be discussed in detail below.

In another particularly preferred embodiment, the data acquisition unit is powered, in particular via a battery. A battery is, for example, an accumulator. In this embodiment, the data acquisition unit can actively manage the storage and/or further processing (with one or more processors and memory) of acquired patient data. This distinguishes the data acquisition unit according to this embodiment from passive data interfaces that merely forward data. Powering the data acquisition unit via a battery is advantageous in that it provides fail-safe operation if an external power supply is interrupted. In a particularly advantageous variant, the data acquisition unit is supplied with power both via a battery and via a power supply unit with a connection to an external power supply. This ensures that the data acquisition unit functions particularly reliably.

In a further embodiment, an analog/digital conversion of at least part of the determined patient data takes place within one or both physiological sensors and/or within at least one adapter cable of the adapter cables, preferably for one or more respectively assigned physiological sensors. In this embodiment, it can be ensured that the data received by the data acquisition unit is already available in digital form, wherein both the measurement signal and the reference signal can be received by the data acquisition unit. It is possible to digitize only the measurement signal or all measurement signals and make them available to the data acquisition unit and to make the reference signal available to the data acquisition unit in analog form. Examples of an implementation of such an analog/digital conversion, but without the described exchange and/or forwarding and/or provision of the reference signal, are familiar to the person skilled in the art and are therefore not explained in detail below. By way of example, reference is made here to the publication DE 10 2016 005 324 A1 and corresponding publication US 2017/316671 AA (which are incorporated by reference).

In a further advantageous embodiment, the at least one slot of the data acquisition unit is configured as a USB slot with an additional pin for the reference signal. The provision of a USB slot advantageously enables the use of known transmission protocols and thus a cost-effective production of the data acquisition unit. Preferably, a further slot is provided in addition to the at least one USB slot (port). The additional slot can be configured to receive the reference signal in addition to or as an alternative to the additional pin for the reference signal

The slot or the additional slot is a component of the corresponding plug connection, wherein this component can be the female or the male part of the corresponding plug connection.

In a particularly preferred embodiment, at least one sensor from the plurality of sensors is formed from a patient cable with a plurality of electrodes, in particular with three or four electrodes. This patient cable can be supplemented by a further sensor for a lead with a plurality of electrodes. The data acquisition system according to the invention is particularly advantageous for use with a patient cable according to this embodiment. For example, the advantageous use of sensor groups makes it possible to connect different patient cables via a corresponding plug connection. In this way, the number of leads of the corresponding measurement signal can be adapted depending on the current situation of the patient. Thus, the modularity of the data acquisition system according to the invention ensures a corresponding modularity of the examination carried out. In addition, the data acquisition system in this embodiment can save material for the patient cables, as not every patient cable has to be routed to a common connector. Since patient cables are often disposed of after a single use, waste can be avoided by using shorter cables.

In a particularly preferred variant of the preceding embodiment, the sensor group comprises at least two patient cables connected to each other via the plug connection, so that an (n+m−1) lead measurement signal and a reference signal, i.e. an (n+m) lead, can be acquired by the data acquisition system from a first patient cable with n electrodes and a second patient cable with m electrodes. This variant advantageously shows how the change in the tree structure of cables made possible by the data acquisition system according to the invention enables a change in the lead of the measurement signal and the reference signal. In this way, the examination carried out can always be adapted to the patient's current treatment situation. In particular, the number of leads can be changed when the patient leaves the intensive care unit, for example. In order to combine the corresponding patient potentials of the measurement signals with each other, the slots used preferably have a transmission channel for the existing neutral potential in addition to the USB lines (5V, GND, D+, D−). It is also conceivable that data can be transmitted alternatively or additionally via SPI, I²C, UART, RS232, LAN, WLAN or Bluetooth

In an advantageous example, a patient cable with three electrodes is combined with another patient cable with three electrodes to enable a 6-fold lead. In this example, the two patient cables form a sensor group connected to each other via a plug connection. This sensor group can, for example, be extended by a patient cable with four electrodes to form a sensor group consisting of three patient cables to provide a measurement signal with a 9-fold lead and the reference signal, i.e. a total of a 10-fold lead. Depending on the course of treatment, this sensor group can be reduced again by one or more patient cables in order to provide a reduced lead during the examination. In accordance with the invention, this adaptation of the lead of the signals can be carried out without having to make any changes to the direct plug connections of the data acquisition unit and/or a hub of the data acquisition unit. The plug connections with which the sensors and/or adapter cables for a respective sensor can be directly connected to each other to reduce the workload in everyday clinical practice. This means that cables can be connected to the data acquisition unit or a corresponding hub without the need for time-consuming reconnection (re-plugging). The tree structure of cables can be changed quickly and easily.

According to a second aspect of the invention, a process for acquiring patient data of a patient is proposed for solving the above-mentioned task, comprising the steps of:
a. Providing a plurality of physiological sensors with a plurality of electrodes to collect patient data;
b. Signal connecting at least two physiological sensors and/or adapter cables for a respectively assigned physiological sensor with each other directly via a plug connection in order to form a sensor group;
c. Output of the patient data determined by the sensor group to a data acquisition unit via a common connector,
   wherein the common connector connects the sensor group directly to the data acquisition unit, the sensor group to a hub for the data acquisition unit and/or the sensor group to an adapter cable for the data acquisition unit.

The process according to the invention is carried out by the data acquisition system according to the first aspect of the invention and comprises the explained advantages of this data acquisition system. In particular, the process according to the invention allows a particularly simple and clearly structured tree structure of cables in the immediate vicinity of the patient by connecting sensors and/or adapter cables for a respective sensor to form a sensor group. This can reduce the effort involved in connecting the patient to a corresponding data acquisition system. This also helps to avoid errors when preparing the plug connections for the data acquisition system.

Preferably, the process according to the invention is carried out in the order indicated. Individual steps, such as the signal connection of at least two physiological sensors and/or of adapter cables for a respectively assigned physiological sensor, can preferably be carried out manually.

In a particularly preferred embodiment, a final step of the process according to the invention comprises a wireless output of the collected patient data to a database. In this embodiment, the data acquisition unit enables centralized communication with the database for storing and managing the patient data. This avoids the existence of a plurality of interfaces with the database. Preferably, communication with the database takes place via a hospital network.

In another particularly preferred embodiment, a prior step comprises a unique assignment of the data acquisition unit to the patient. Preferably, this prior step is carried out via a software protocol. This unique assignment can advantageously reduce the complexity of the tree structure of cables in the patient's environment, since the data acquisition unit is not connected to cables from several patients. In a preferred variant of this embodiment, only one data acquisition unit is advantageously assigned to one patient. In this way, the data acquisition unit can form a central location for collecting, managing and/or further processing patient data of a specific patient.

In a further embodiment, an analog/digital conversion takes place for at least part of the determined patient data between the determination at the respective physiological sensor and the reception by the data acquisition unit. The analog/digital conversion allows the patient data to be easily processed digitally by the data acquisition unit.

In a further advantageous embodiment, the step of signal connecting at least two physiological sensors and/or adapter cables for a respectively assigned physiological sensor using signal technology is carried out several times in order to form a sensor group comprising more than two physiological sensors and/or in order to form several sensor groups. In this embodiment, several plug connections are advantageously used for the respective grouping of sensors and/or adapter cables for a respective sensor. By using several plug connections between sensors and/or adapter cables, the tree structure of cables on the patient can be advantageously optimized, i.e. in particular the complexity of the tree structure can be reduced.

The invention will now be explained in more detail with reference to advantageous embodiments shown schematically in the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic representation of a first embodiment of a data acquisition system according to a first aspect of the invention;

FIG. 2 is a schematic representation of a second embodiment of the data acquisition system according to the first aspect of the invention;

Fig, 3 is a schematic representation of a third embodiment of the data acquisition system according to the first aspect of the invention showing a patient cable;

FIG. 4 is a schematic representation of the third embodiment of the data acquisition system according to the first aspect of the invention wherein a patient cable is combined with a second patient cable;

FIG. 5 is a schematic representation of the third embodiment of the data acquisition system according to the first aspect of the invention wherein a patient cable is combined with a second patient cable and with a third patient cable; and

FIG. 6 is a flowchart of an embodiment of a process according to a second aspect of the invention.

### DESCRIPTION OF PREFERRED EMBODIMENTS

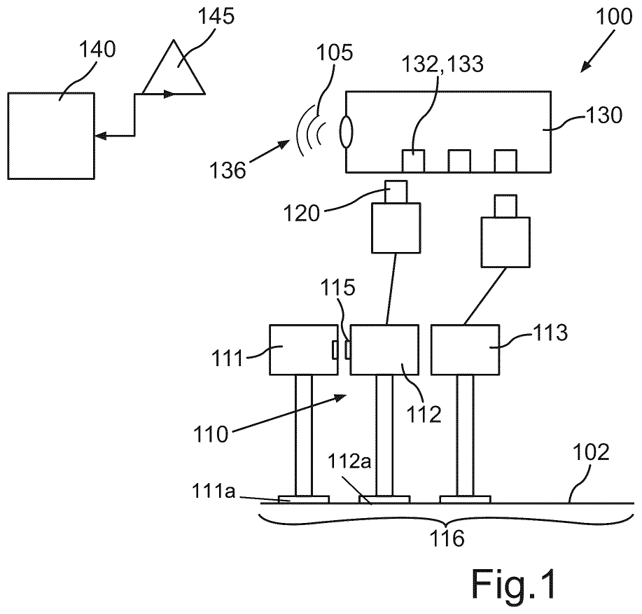

Referring to the drawings, FIG. 1 shows a schematic representation of a first embodiment of a data acquisition system 100 according to a first aspect of the invention.

The data acquisition system 100 is configured to acquire patient data 105, in particular ECG data, of a patient 102. For this purpose, it has a plurality of physiological sensors 110 and a data acquisition unit 130.

The plurality of sensors 110 are configured to determine the patient data 105. In the embodiment example shown, these are two physiological sensors 111, 112 for determining patient data 105 by means of electrodes 111a, 112a and a third sensor 113 for measuring a temperature of the patient 102. The electrodes of these three sensors 111, 112 and 113 are all located on the skin of the patient 102 and are exemplary of a plurality of known sensors. According to the invention, at least two physiological sensors 111, 112 are arranged and configured to be signal connected to each other directly via a plug connection 115, thereby forming a sensor group 116. One or more measurement signals and/or a reference signal can be exchanged and/or forwarded and/or provided via the plug connection 115. The three sensors 111, 112 and 113 may output analog signals. The sensors 111, 112 with the plug connection 115 may include a processor and memory and may multiplex the two or more measurement signals for downstream demultiplexing. The three sensors 111, 112 and 113 may include analog to digital converters or otherwise output digital signals. The sensors 111, 112 with the plug connection 115 may include a processor and memory and may multiplex the two or more digital measurement signals for downstream demultiplexing. The sensors 111, 112 with the plug connection 115 may include a processor and memory to packetize the data with addressing, for downstream handling, and depacketizing. There may be a mix of a digital signal and analog reference signals on a separate signal path, for the digital signal output as separate data packets, such as described in US 2017/316671 AA. Alternatively, or additionally, at least two adapter cables for a respectively assigned physiological sensor can also be arranged and configured to be connected to each other directly signal connected via a plug connection and thereby form a sensor group. Such adapter cables are shown, for example, in FIGS. 3, 4 and 5.

The data acquisition unit 130 is configured to have at least one slot (a port—slot/socket or plug) 132 for a plug connection 133 as a patient data interface. In FIG. 1, three slots 132 of the data acquisition unit 130 are shown as examples. The slots can be parts of a manufacturer-specific plug connection and/or plug connections that are common on the market and compatible with other devices. For example, the slot may be a USB slot. The data acquisition unit 130 may have one or more processors and a memory unit and may be configured to demultiplex analog and/or digital signals and may be configured to handle data packets.

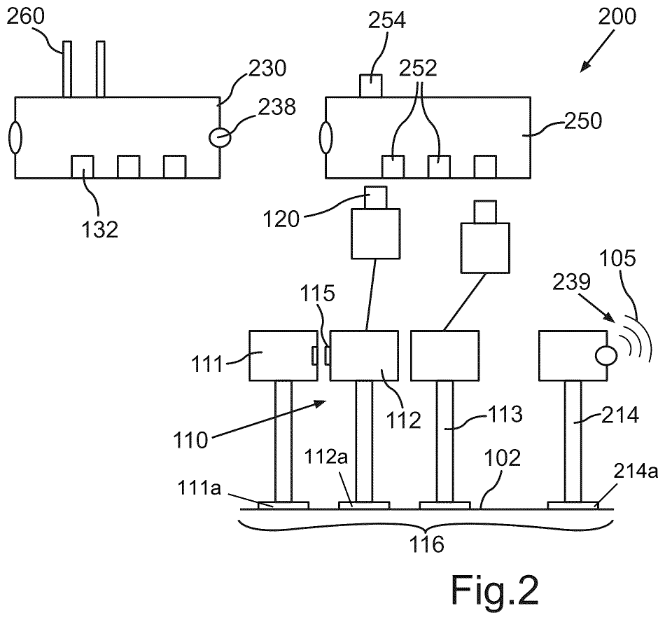

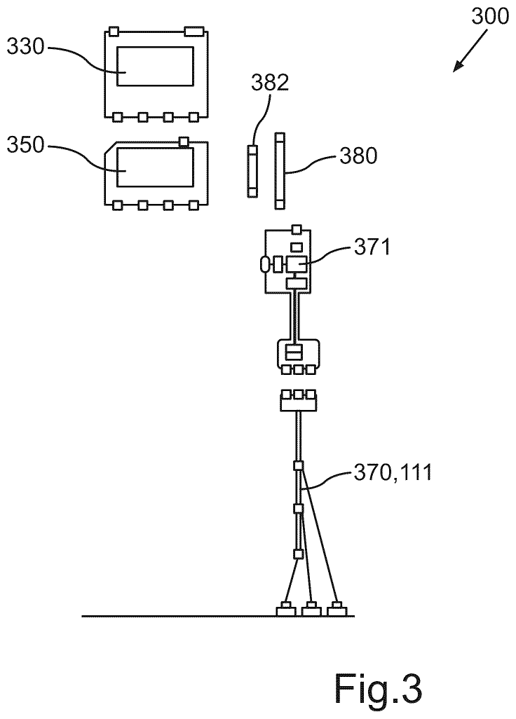

According to the invention, the patient data 105 determined by the sensor group 116 is output to the data acquisition unit 130 via a common connector 120. Thus, in the illustrated embodiment example, the patient data 105 determined by the sensor 111 and by the sensor 112 are output via the common connector 120, the patient data 105 having previously reached the connector 120 from the sensor 111 via the plug connection 115. The measurement signal(s) and the reference signal can be combined or transmitted separately as noted above. In the present case, the output via the common connector 120 is to take place via a direct plug connection with the data acquisition unit 130. Alternatively, the common connector can also be connected to the data acquisition unit 130 via a hub, as shown in FIG. 2, or via an adapter cable, as shown in FIG. 3. In embodiments not shown, several data collectors are provided in the data acquisition system, in particular combined via a corresponding plug connection.

For reasons of clarity and to simplify the illustration, the plug connections are shown unconnected in the illustrated examples.

The third sensor 113 is not part of the sensor group 116, but is connected to the data acquisition unit 130 via a separate connector. The slots 132 provided by the data acquisition unit 130 may be identical or at least partially different.

In the illustrated embodiment example, the data acquisition unit is further configured to output the acquired patient data 105 to a database 140 via a wireless data connection 136. In the present case, this output takes place via a hospital network 145, which is connected to the database 140. Communication with the hospital network preferably takes place via Bluetooth,

WLAN or the like. Alternatively, this output can also be made directly to the database or a corresponding external device. The database 140 is preferably a part of the data acquisition system 100, but in other embodiments according to the invention it is also present as an external database

FIG. 2 shows a schematic representation of a second embodiment of the data acquisition system 200 according to the first aspect of the invention.

The data acquisition system 200 is similar to the data acquisition system 100 shown in FIG. 1, wherein the data acquisition system 200 differs from the data acquisition system 100 in that, among other things, the data acquisition unit 230 of the data acquisition system 200 is further configured to receive patient data 105 from at least one physiological sensor 214 of the plurality of physiological sensors 110 via a wireless communication link 239. Preferably, in addition to the wireless communication link 239 with the sensor 214, a data interface 238 of the data acquisition unit 230 also allows wireless communication with other sensors not shown from the plurality of sensors 110. The physiological sensor 214 has an electrode 214a.

In addition, unlike the data acquisition system 100 of FIG. 1, the data acquisition system 200 has a hub 250, which can be connected to the data acquisition unit 230 via the at least one slot 132. The hub 250 is configured to provide a respective plug connection with the sensor group 116, with a physiological sensor 113 and/or with the adapter cable for a respectively assigned physiological sensor via a plurality of slots 252, namely in the present case via two slots 252. The hub 250 also has a hub connector 254 in order to be able to be connected to a slot 132 of the data acquisition unit 230. As shown in FIG. 2, the hub 250 may be provided as a compact box with a housing. Alternatively, or additionally, the hub may be configured as a signaling link of a plurality of cables or the like. Advantageously, the hub 250 allows an increase in the number of slots available for the data acquisition unit 230. In addition, the hub 250 can increase an application range of the data acquisition unit 230 by means of special types of slots. In particular, plug connections that have not yet been provided for the data acquisition unit 230 can also be used for acquiring the patient data 105 according to the invention by retrofitting with a corresponding hub.

Finally, the data acquisition unit 230 of the data acquisition system 200 is also supplied with power, in particular supplied with power via a battery not shown. In addition to the battery, a mains (grid power/AC power) connection 260 is also provided on the data acquisition unit 230. The power supply can be provided via the general mains voltage and/or via Power over Ethernet. This provides the energy required for the operation of the data acquisition unit 230 in a particularly safe and reliable manner.

An analog-to-digital conversion preferred for further processing of the patient data 105 obtained at the plurality of sensors 110, in particular the patient data 105 generated from a plurality of physiological sensors with the sensor group 116, preferably takes place within the respective sensor 111, 112, 113, 214, and/or within the adapter cable for one or more respectively assigned physiological sensor(s) 111, 112, 113, 214. This ensures that the data acquisition unit 230 preferably directly receives digital patient data 105 and can forward and/or store it. In an embodiment example not shown, the analog/digital conversion of the patient data 105 only takes place in the data acquisition unit 230.

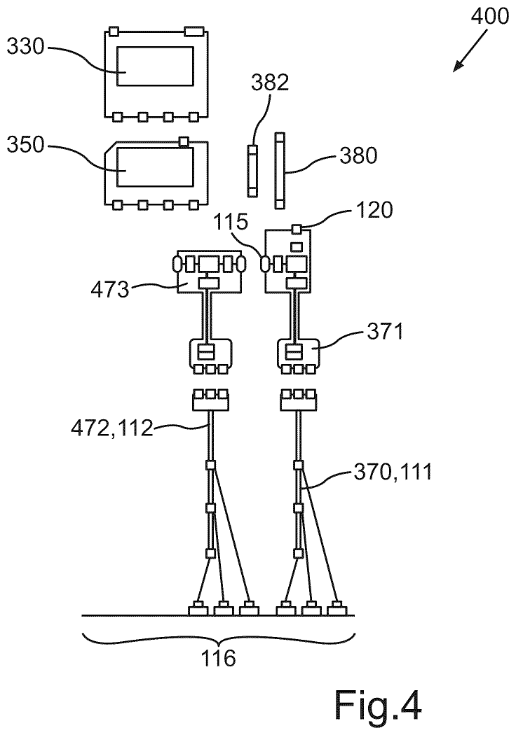

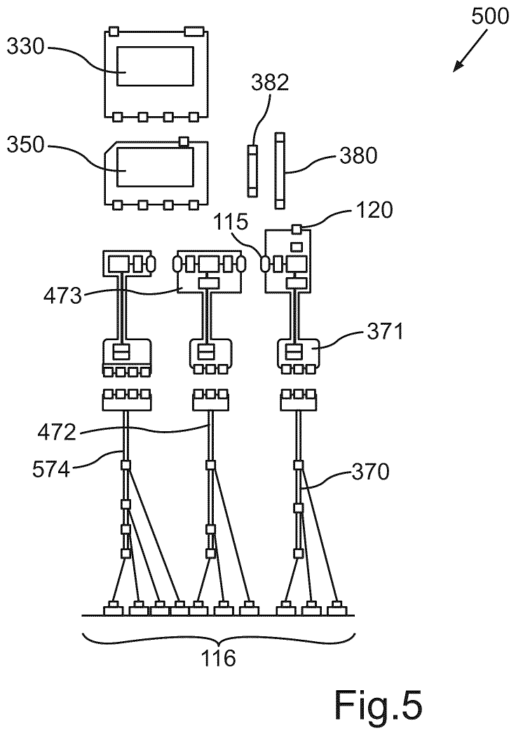

FIGS. 3, 4 and 5 show a schematic representation of a third embodiment of the data acquisition system according to the first aspect of the invention, wherein a first patient cable (FIG. 3) is combined with a second patient cable (FIG. 4) and with a third patient cable (FIG. 5).

The use of a single patient cable 370 for the data acquisition system 300 according to the invention, as shown in FIG. 3, only involves the use of a sensor 111 in the form of the patient cable 370 with three electrodes.

This arrangement therefore enables the acquisition of a 3-lead ECG measurement signal.

An adapter cable 371 is directly associated with the patient cable, which in the case shown also acts as an analogue/digital converter to digitize the determined patient data 105.

Since such patient cables are typically combined with such an adapter cable 371, this adapter cable 371 can also be present as part of (integrated with) the patient cable.

The combination of sensors 111, 112 to form a sensor group 116 according to the invention is shown in FIG. 4, in which the data acquisition system 300 of FIG. 3 is supplemented by a second patient cable 472 to the data acquisition system 400

In this case, the second patient cable 472 is connected to the first patient cable 370 by a plug connection 115 via the corresponding adapter cables 371, 473, so that these two patient cables 370, 472 form the sensor group 116 and can be connected to the data acquisition unit 330 via a common connector 120. As an alternative to the direct connection, FIGS. 3, 4 and 5 also show two different additional adapter cables 380, 382 and the hub 350, which can be interposed between the common connector 120 and the data acquisition unit 330.

The combination of the two patient cables 370, 472 ensures that the number of leads can be increased to a 5-lead measurement signal with a reference signal, i.e. to a 6-fold lead, by simply plugging in a patient cable.

FIG. 5 shows an addition of a further patient cable 574 to the data acquisition system 400. This forms a sensor group 116 consisting of three patient cables 370, 472, 574. This further increases the number of leads of the corresponding signals to a corresponding 10-fold lead.

The exact arrangement of the respective electrodes of a patient cable is shown only ideally in FIGS. 3, 4 and 5.

In principle, the data acquisition system according to the invention can have several hubs, several adapter cables and/or other interconnected electronic components. These components can be combined and connected in series for individual or multiple sensors.

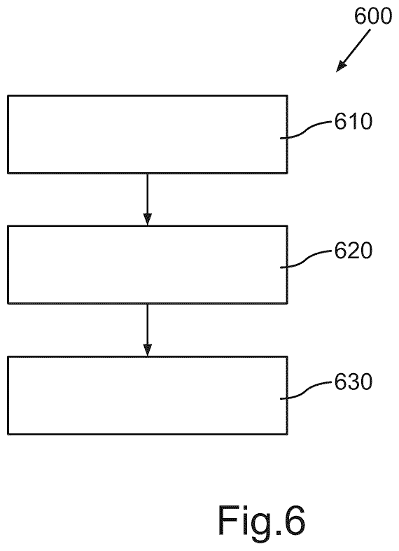

FIG. 6 shows a flow diagram of an embodiment of a process 600 according to a second aspect of the invention.

The process 600 according to the invention is configured to acquire patient data 105 of a patient. For this purpose, the process 600 has the steps explained below.

A first step 610 comprises providing a plurality of physiological sensors having a plurality of electrodes for determining patient data 105.

A subsequent step 620 comprises a signal connection of at least two physiological sensors 111, 112 and/or adapter cables 371, 473 for a respective associated physiological sensor 111, 112 to each other directly via a plug connection 115 to form a sensor group 116.

A further step 630 comprises outputting the patient data 105 determined by the sensor group 116 to a data acquisition unit 130 via a common connector 120, wherein the common connector 120 connects the sensor group 116 directly to the data acquisition unit 130, the sensor group 116 to a hub 250 for (signal connected to) the ta acquisition unit 130, and/or the sensor group 116 to an adapter cable 371 for (signal connected to) the data acquisition unit 130.

Preferably, the process steps 610, 620 and 630 are carried out in the order indicated. At least step 620 is preferably carried out manually.

The process 600 according to the invention is preferably finally supplemented by a wired or wireless output of the acquired patient data 105 to a database 140. Alternatively, or additionally, the acquired patient data 105 can also be stored in a memory unit of the data acquisition unit 130 or in another device connected to the data acquisition unit 130, such as a patient monitor.

In a particularly advantageous embodiment, a step preceding the process 600 comprises uniquely associating the data acquisition unit 130 with a patient. Preferably, there is also a unique assignment of the patient to the data acquisition unit 130, so that during a hospital stay all relevant physiological patient data 105 of a patient is collected via this data acquisition unit 130. This may be done by providing the data acquisition unit 130 with unique patient data to associate one patient with the data acquisition unit for physiological patient data during a medical treatment period (a hospital stay).

Not shown in FIG. 6 is an analog/digital conversion of the determined patient data 105, which preferably takes place between the determination of data at the respective sensor 111, 112 and the reception of data by the data acquisition unit 130.

In a preferred embodiment of the process 600, the step of signal connecting at least two physiological sensors 111, 112 and/or adapter cables 371, 473 for a respective associated physiological sensor 111, 112 is performed multiple times to form a sensor group 116 comprising more than two physiological sensors 111, 112 and/or to form multiple sensor groups 116. This addition to the process has to be carried out, for example, in the context of providing the data acquisition system 500 in FIG. 5, since the sensor group 116 therein comprises more than two sensors.

While specific embodiments of the invention have been shown and described in detail to illustrate the application of the principles of the invention, it will be understood that the invention 10 may be embodied otherwise without departing from such principles.

### LIST OF REFERENCE SYMBOLS

100, 200, 300, 400, 500 Data acquisition system
102 Patient
105 Patient data
110 Plurality of physiological sensors
111, 112, 113, 214 Physiological sensor
111a, 112a, 214a Electrode
115 Plug connection to form a sensor group
116 Sensor group
120 Common connector of a sensor group
130, 230, 330 Data acquisition unit
132 Slot of the data acquisition unit
133 Plug connection of the data acquisition unit
136 Wireless data connection
140 Database
145 Hospital network
238 Data interface for wireless communication
239 Wireless communication connection
250, 350 Hub
252 Slot (port—slot/socket or plug) of the hub
254 Hub connector
260 Mains connection
370, 472, 574 ECG (EKG) patient cable
371, 473 Adapter cable
380, 382 Further adapter cable
600 Procedure
610, 620, 630 Process steps

What is claimed is:

1. A data acquisition system for acquiring patient data of a patient, the data acquisition system comprising:
a plurality of physiological sensors for determining patient data by means of electrodes, wherein at least two of the plurality of physiological sensors and/or adapter cables respectively associated with the at least two of the plurality of physiological sensors are arranged and configured to be signal connected to one another directly via a plug connection to form a sensor group, which sensor group and/or the plug connection is configured to provide a measurement signal and a reference signal of the electrodes; and
a data acquisition unit, which is configured to comprise at least one slot for a plug connection as a patient data interface; and
a common connector configured to output the patient data determined by the sensor group to the data acquisition unit, the common connector being configured for a direct plug connection with the data acquisition unit, for a plug connection with a hub signal connected to the data acquisition unit and/or for a plug connection with an adapter cable signal connected to the data acquisition unit.

2. A data acquisition system according to claim 1, wherein the data acquisition unit is configured to output the acquired patient data to a database via a wireless data connection.

3. A data acquisition system according to claim 1, wherein the data acquisition unit is configured to receive patient data from at least one physiological sensor of the plurality of physiological sensors via a wireless communication link.

4. A data acquisition system according to claim 1, further comprising the hub, wherein the hub is configured to be connected to the data acquisition unit via the at least one slot and configured with a plurality of slots to provide a respective plug connection for connection to the sensor group, to a physiological sensor and/or to the adapter cable for a respective associated physiological sensor.

5. A data acquisition system according to claim 1, wherein the data acquisition unit is powered via a battery.

6. A data acquisition system according to claim 1, wherein one or both of the at least two of the plurality of physiological sensors and/or the at least one adapter cable of the adapter cables is configured for an analog-to-digital conversion of at least a part of the determined patient data.

7. A data acquisition system according to claim 1, wherein the at least one slot of the data acquisition unit is a USB slot.

8. A data acquisition system according to claim 1, wherein at least one sensor of the plurality of sensors comprises a patient cable with a plurality of the electrodes.

9. A data acquisition system according to claim 8, wherein the sensor group comprises at least two patient cables connected to each other via the plug connection, such that an n+m−1 measurement signal and a reference signal can be acquired by the data acquisition system from a first patient cable with n electrodes and a second patient cable with m electrodes.

10. A data acquisition system according to claim 1, wherein the data acquisition unit is assigned unique patient data to associate physiological patient data of a patient with the data acquisition unit for a medical treatment period.

11. A process for acquiring patient data of a patient, the process comprising the steps of:
providing a plurality of physiological sensors with a plurality of electrodes for determining patient data;
signal connecting at least two physiological sensors of the plurality of physiological sensors and/or adapter cables for a respectively assigned physiological sensor to one another directly via a plug connection to form a sensor group;
outputting the patient data determined by the sensor group to a data acquisition unit via a common connector;
with the common connector, connecting the sensor group directly to the data acquisition unit, connecting the sensor group to a hub for the data acquisition unit and/or connecting the sensor group to an adapter cable for the data acquisition unit.

12. A process according to claim 10, further comprising a subsequent step of wirelessly outputting the acquired patient data to a database.

13. A process according to claim 10, further comprising a prior step of uniquely associating the data acquisition unit with the patient.

14. A process according to claim 10, further comprising providing an analog-to-digital conversion of at least a part of the data determined at the respective physiological sensor and data received by the data acquisition unit.

15. A process according to claim 10, wherein the step of signal connecting at least two physiological sensors and/or adapter cables for respective associated physiological sensors is performed several times to form a sensor group of more than two physiological sensors and/or to form several sensor groups.

16. A process according to claim 10, wherein the data acquisition unit is configured to output the acquired patient data to a database via a wireless data connection.

17. A process according to claim 10, wherein the data acquisition unit is assigned unique patient data to associate physiological patient data of a patient with the data acquisition unit during a medical treatment period.
